(19) United States
(12) Patent Application Publication
(10) Pub. No.: US 2004/0063165 A1
(43) Pub. Date: Apr. 1, 2004

(54) Light-activated in vitro assay process for luciferase bioluminescence

(76) Inventor: Yahia A. Gawad, Mississauga (CA)

Correspondence Address: Dinesh Agarwal, Law Office, Suite 330, 5350 Shawnee Road, Alexandria, VA, 22312, US

(21) Appl. No.: 10/450,256

(22) PCT Filed: Dec. 14, 2001
(86) PCT No.: PCT/CA01/01787

(51) Int. Cl. C12Q 1/66

(52) U.S. Cl. 435/8

(57) ABSTRACT

There is provided a process of inducing luminescent emission from a luciferase bioluminescent reaction particularly useful in binding assays. A luciferase bioluminescent combination, together with an inactive, caged trigger compound such as a cofactor, is subjected to photonic radiation, so as to release the trigger compound in active form, and thereby cause substantially instantaneous reaction of the active trigger compound so released with the luciferase combination, to induce photonic emission which can be detected and measured.

### FIELD OF THE INVENTION

This invention relates to chemiluminescent processes and reactions, and the use thereof in bioanalytical assays. More specifically, it relates to bioanalytical assays such as immunoassays and nucleic acid hybridization assays, which involve bioluminescence of a luciferase enzyme as an indicator of the presence and quantity of a target analyte in a test fluid.

### BACKGROUND OF THE INVENTION

Classically, substances are detected in liquids based on a reaction scheme wherein the substance to be detected is a necessary reactant, the presence of which is usually indicated by the appearance of a reaction product or the disappearance of a known reactant. The high specificity of binding in many biochemical and biological systems has led to the development of many assay methods and systems based upon the well-known binding reactions. Binding reactions based on the principles of bioaffinity and/or enzymatically catalyzed reactions have been developed in order to analyze, detect and quantify important compounds from biological and environmental sources. These bioaffinity binding assays have become invaluable tools in research and clinical diagnostics in a great variety of formats using various detection principles.

Various assay methods and systems, including immunoassays, receptor-ligand binding assays and probe hybridization assays are known for obtaining desired chemical and biological information. These binding assays rely on specific bioaffinity recognition reactions in which natural biological binding components (such as antibodies, natural hormone binding proteins, lectins, enzymes, receptors, DNA, RNA or peptide nucleic acids (PNA)) or artificially produced binding compounds (such as genetically or chemically engineered antibodies, or nucleic acid probes) form the specific binding partner to the analyte under investigation. Such assays generally rely on a label to quantify the binding product after suitable separation form excess reag nts.

According to the assay design and specific needs, many labeling technologies are applied. Assays can utilize a simple labeled substrate, which facilitates the measurement of either substrate or end product, or it may be defined in such a way as to give direct information of hydrolysis (e.g. internal quenching or energy transfer).

Use of luminescent labels is well recognized in the field of bioaffinity assays. Luminescence is the term applied to emission of light by a substance (other than by incandescence). Luminescent labels can be made to luminesce through photochemical, chemical, and electrochemical means. “Photoluminescence” is the process whereby a material is induced to luminesce upon absorbing electromagnetic radiation. Fluorescence and phosphorescence are types of such photoluminescence. Chemiluminescence is a process that entails the creation of luminescent species by the chemical transfer of energy during a chemical reaction in the form of a single photon. Chemiluminescent labels are well recognized in the field of bioassays. The chemiluminescent emission is generally of sufficient duration to enable the emitted light to be detected or measured, and thereby to allow the detection or quantification of an analyte.

Bioluminescence is a type of chemiluminescence where one of the components of the chemiluminescent reaction is of biological origin. It is known to use luminescence, i.e. chemiluminescence or bioluminescence, as the signal-generating mechanism in immunoassays as well as in most other bioaffinity binding assays. Bioluminescence arises from the oxidation of an organic molecule, the “luciferin”, by oxygen or one of its metabolites, in the presence of a catalyst, which may be a true enzyme (luciferase) or a non-enzymatic protein (photoprotein) with or without the presence of cofactors resulting in the production of light. In the case of photoprotein-catalyzed luminescent reactions, the luciferin is so bound to the photoprotein that the catalyzed reaction results in the emission of light without release of the oxyluciferin into the reaction medium. In the case of luciferase-mediated bioluminescent reactions, the luciferin is not tightly bound to the luciferase and the catalyzed reaction results in the oxidation of a luciferin substrate and the dissociation of the formed oxyluciferin from the complex into the medium with the resultant emission of light.

A large number of bioluminescent reactions that utilize several types of luciferases are known. Luciferases mediating these luminescent reactions are numerous and diverse. Luciferases most commonly used in binding assays to generate bioluminescence are those of firefly, bacterial and renilla or sea pansy origin. Each of these luciferases utilizes a different luciferin and different cofactors. The firefly luciferase utilizes adenosine triphosphate (ATP) as its cofactor for the oxidation of its luciferin and the emission of light. Bacterial luciferases use nicotinamide-adenine dinucleotide phosphate (NAD(P)) or flavin mononucleotide (FMN) as co-factors for the oxidation of their luciferins to generate a luminescent reaction.

### THE PRIOR ART

There is extensive prior art on the use of luciferases in binding assays taking advantage of the chemiluminescent reactions. Several groups have coupled the luciferases of firefly, bacteria or renilla to antibodies and have used them in binding assays. For example, U.S. Pat. Nos. 5,283,179, 5,641,641 and 5,650,289 have been issued to Promega Corporation for firefly luciferase binding assay methods. These assays are commercially available, and through their use, the power of bioluminescence has been demonstrated.

As a general rule in the extensive prior art, a bioluminescent assay requires addition of several chemical reagents in a particular sequence in order to trigger the final reaction of light generation. As the final step in all bioluminescent assays, a trigger compound is added to all the other reaction components at a particular moment in order to induce the light generating reaction.

Although the advantages of bioluminescence in binding assays are well recognized, automation of such assays is complicated by the numerous steps involved in triggering such reactions. Manual or automatic injectors are needed to supply reagents at particular reaction steps. These requirements have hampered the widespread use of bioluminescent assays. Although instruments with automated injectors have been developed and are currently available, instrumentation is mostly manually operated.

Due to the time course of light emission, a body of art has been developed either to prolong the period of light emission or to deliver the trigger compounds at a precise instant in order to maximize the light emission and collection. Specifically, when multiple samples are processed, it is essential to deliver the trigger compound at an appropriate time so that the light detection is maximized. Typically, luciferase-catalyzed photon production ceases within few seconds and due to the nature of this flash of emitted light, bioluminescent assays capturing this short flash have limited reliability. Also, due to the imprecision of the mechanical means used to deliver the components of the bioluminescent reaction, the high variability of these assays necessitates expensive machinery. In addition, the many steps involved in adding chemical reagents to the bioluminescent reaction mixture make automation of these reactions difficult and necessitate the development of highly sophisticated and very expensive machinery. Furthermore, although catalytic turnover of the luciferase would be expected to yield a steady luminescent intensity, in practice the enzyme assay generates only a brief burst of light upon addition of substrates. In assays using bacterial luciferases, reduced FMN is rapidly auto-oxidized in aqueous solutions and thus is unavailable for sustained catalysis. This is one of the reasons that bacterial luciferases are not generally preferred as bioluminescent reagents over the alternatives, most notably firefly luciferase.

In attempting to address some of these shortcomings, an extensive prior art has been developed to optimize luciferase reaction performance. Attempting to improve the firefly luciferase assays, several of the previously identified patents assigned to the Promega Corporation reported improved kinetics of light emission in order to afford greater light output to optimize these assays and maximize their sensitivity as compared to the conventional assay. U.S. Pat. No. 4,286,057 teaches the incorporation of adenosine 5′-monophosphate (AMP), adenosine diphosphate (ADP), ethylenediamine tetraacetic acid (EDTA), sulfhydryl compounds and albumin into a reagent composition. This reagent composition extends the duration of the light flash of the firefly luciferase in order to optimize period of light emission upon the addition of ATP. Furthermore, the same reagent composition has been utilized to improve the light emission of the renilla luciferase as disclosed in PCT patent application 99/38999.

In an attempt to protect ATP from premature hydrolysis and to optimize ATP delivery to the reaction medium, Bernstein discloses in U.S. Pat. No. 4,704,355 a method for using liposome-encapsulated ATP in triggering firefly luciferase bioluminescence during binding assays. Also, in U.S. Pat. No. 5,786,151, Sanders discloses another method of liposomal encapsulation of ATP during a bioluminescent assay that utilizes firefly luciferase. Although such encapsulation and assays provide a very sensitive process for detecting the presence of analytes such as antigens and DNA probes, the encapsulation is achieved within liposomes, which require chemical hydrolysis before release of ATP. This process is slow and results in the slow leakage of ATP into the reaction medium over time thereby resulting in weak light emission over an extended period.

However, there still remain significant shortcomings with the current bioluminescent binding assays, especially when multiple samples need processing. Inappropriate extension of the period of light emission may interfere with the signal measurement of other samples. Also, optimizing assay reagents can interfere with the assay medium. As one of the major limitations in using the different luciferases in bioluminescent binding assays is the short duration of photon production, the addition of the trigger reagent has to be done when the reaction components are within the measuring chamber of the detector. Furthermore, suitable mixing of the reagents is essential for consistent light production with these reagents. Means for extending the period of photon production or for optimizing the kinetics of light generation have been eagerly sought.

The development of technologies to allow these chemical reactions to be automated would simplify the assay procedures. Furthermore, precise availability of the reagents will maximize the detection of light.

It is an object of the present invention to provide a novel process for conducting bioluminescent reactions using luciferases.

It is a further and more specific object of the invention to provide a process whereby such luciferase-based bioluminescent reactions are applied to in vitro bioluminescent binding assays.

It is a further object to provide a novel composition of matter for use in bioluminescent reactions.

### SUMMARY OF THE INVENTION

The present invention provides a novel method for inducing the bioluminescent reaction of the luciferase enzymes and their associate luciferins useful for carrying out binding assays. The method of the invention is particularly suitable for carrying out in vitro bioassay processes, but also has utility in other technical areas.

The process utilizes the photochemical release of a trigger compound in active form, from an inactive, caged form, as the final reagent needed in a luciferase-mediated bioluminescent reaction. Caged compounds are molecules whose biological function is masked until radiation such as an UV light pulse induces a photochemical reaction that converts the molecules an inactive into an active state. The activation of these compounds can be precisely controlled both temporally and spatially by limiting their exposure to light. By inducing a light-driven release of a trigger compound, (such that the trigger compound is released substantially instantaneously and interacts with all other components required for a luminescence-generating luciferase reaction that are already present), a detectable and measurable luminescent signal is reproducibly generated and easily detected. Initially, the trigger compound is present in a photolabile caged form, which is inactive. Upon subjection of this inactive precursor compound to a very short pulse of photonic energy of appropriately chosen and predetermined characteristics the trigger compound is released in its active form.

The process of the invention allows for close and strict control over the initiation of the bioluminescence reaction. By having full control over the introduction into the reaction mixture of the active form of the trigger compound, both with regard to time of generation and the quantity of generation thereof, this method allows for optimizing the amount of the released trigger compound into the reaction medium and therefore the extent of the light flash developed during the bioluminescent assay. It allows for the use of a variety of different trigger compounds, chosen on the basis of their specificity for bioluminescent reaction with the chosen luciferase.

The method of the invention involves the photorelease of a defined trigger compound to initiate a bioluminescent reaction of a luciferase enzyme by supplying the essential cofactor needed to initiate the reaction, by photonic release from an inactive caged compound. It also allows for control over the kinetics of the generation of photons by a luciferase enzyme in such binding assays. All these features allow the operator to maximize the sensitivity of the process, such as by amplifying the emitted luminescent signal on the basis of the appropriate choice of luciferase enzyme and its specific trigger compound, and for optimizing the duration of collecting the generated luminescent signal.

Thus, according to the present invention, from one aspect, there is provided a process of inducing luminescent emission from a luciferase bioluminescent reaction mixture which comprises:

preparing a reaction mixture including components required for a specific luciferase bioluminescent reaction, said components including a luciferase, a luciferin chosen for specific interaction with said luciferase, and at least one cofactor therefor, one said component being initially included in the reaction mixture in an inactive, caged form;

subjecting the reaction mixture to photonic radiation of appropriately chosen characteristics to cause release of the one said component from its caged form, in active form thereby triggering the bioluminescent reaction;

and detecting the photonic emission so caused.

The process of the invention allows for very close control over the amount of released trigger compound by varying the amount of photonic energy delivered to release a predetermined amount of the trigger compound and therefore optimize light generation by the bioluminescent luciferin-luciferase reaction.

Another aspect of the present invention comprises a composition of matter including a luciferase-luciferase emitter as defined herein, and a trigger compound which is a photolabile, caged, inactive cofactor adapted to release the cofactor in active form upon appropriate photonic energy input thereto, and being chosen to interact with the luciferase-luciferase emitter to initiate bioluminescence when the cofactor is photonically released in its active form.

### DESCRIPTION OF THE PREFERRED EMBODIMENTS

As the term is used herein, a luciferase is an enzyme, which catalyzes the oxidation of a luciferin to oxyluciferin with the generation of light in a bioluminescent reaction such that the oxyluciferin is released from the luciferin/luciferase complex into the reaction medium. The reaction of these luciferases to generate light requires the presence of one or more cofactor such as ATP, NAD/NADP or FMN. Examples of such luciferases are those of firefly, bacteria and fungi.

As defined herein, photoproteins are proteins that catalyze the oxidation of a luciferin to generate light in a bioluminescent reaction without the release of the formed oxyluciferin from the complex into the reaction medium. Photoproteins utilize divalent cations such as calcium as the sole trigger compound for emitting light. Examples of such photoproteins are aequorin and obelin, which utilize coelenterazines as luciferins and Ca⁺⁺ as the trigger compound. Photoproteins are separate and distinct from luciferases, as the terms are used herein.

The term “luciferin” as used herein refers to any substrate which, upon oxidation by an appropriately chosen luciferase enzyme, produces bioluminescence. Luciferins may be natural or synthetic compounds. Luciferins isolated from different biological species may vary greatly in structure, although in many cases identical structures have been found in widely diverse species. Structurally luciferins can take the form of certain aldehydes, imidazolopyrazines, benzothiazoles, linear tetrapyrroles and flavins. Firefly luciferin, for example, is (S)-4,5-dihydro-2-(6-hydroxy-2-benzothiazolyl)-4-thiazole carboxylic acid, and was originally isolated from active fireflies. It is commercially available. Marine bacterial luciferin is a reduced riboflavin phosphate FMNH₂ Dinoflagellate luciferin, from the genus Gonyaulax, has a chemical structure very similar to that of chlorophyll. Vargulin, the luciferin found in the ostracod Vargula and in the midshipman fish Porychthys, is a benzazole-diazine compound with a terminal alkylene-guanidine group. The best known and most widely studied naturally occurring luciferins are those isolated from th sea pansy, Renilla reniformis, from the ostracod, Cypidina hilgendorfii, form the limpet, Latia neritoides, and from the firefly Photinus pyralis.

Some luciferin-luciferase combinations also need the presence of a cofactor in order to produce bioluminescence on oxidation. Firefly luciferin/luciferase, for example, needs the presence of cofactor ATP for its oxidative bioluminescent reaction.

The luciferase/luciferase emitters combination referred to herein means any combination of a luciferase with its appropriate luciferin in the presence of all other specific cofactors needed to initiate a bioluminescent reaction, except for the trigger compound as defined herein. There are numerous luciferase/luciferase emitters combinations; each luciferase uses a specific luciferin and co-factors. A luciferase/luciferase emitters combination can be any such combination provided that, on introduction of the trigger compound in its active form, a bioluminescent reaction is initiated and light is generated.

A trigger compound is a cofactor needed for the initiation of a luciferase-mediated bioluminescent reaction (other than divalent cations) when added to a luciferin-luciferase combination. Examples of such trigger compound are ATP, NAD/NADP and FMN. A trigger compound is chosen to be capable of interaction with the luciferase/luciferase emitters combination in the presence of the appropriately chosen other components (which may include a multivalent cation) to trigger a bioluminescent reaction.

A photolabile caged compound is an inert derivative of an active molecule, which, when irradiated with a pulse of light of specific energy, is induced to release the active species. The release of the free molecule is essentially instantaneous. A photolabile caged trigger compound may consist of a single component complex such that it is introduced to the luciferase/luciferase emitters combination as an inactive precursor and is activated upon illumination with photonic energy. A photolabile caged trigger can also be a two-component system such that the trigger compound is physically separated from the reaction within a photolabile carrier entity such that a pulse of light of specific energy is induced to release the active trigger compound from the physical barrier. Any one of the essential ingredients for the photoluminescent reaction may be introduced initially as a caged, inactive compound in the process of the invention. Preferably the caged compound is either a caged cofactor or a caged luciferin.

As applied to a heterogeneous binding assay, the method of the present invention utilizes the photochemical release of a trigger compound to initiate a luciferase-mediated bioluminescent reaction as the signal-generation mechanism for such an assay. This photorelease of a trigger compound, which uses photonic energy, provides the final component needed to initiate the bioluminescence reaction. Such trigger compound is not a divalent cation of the type required for photoproteins. In the presence of the other essential components of a luciferase-driven bioluminescent reaction, the release of the trigger compound initiates the luminescent reaction substantially instantaneously causing photonic emission at a wavelength that is specific for each different luciferase reaction. The luciferase enzyme may be attached directly or indirectly to a binding partner, which specifically binds the analyte being assayed, or an analyte analog to compete with the analyte for binding to a specific binding partner so that detection and quantification of the photonic emission as triggered allows detection and quantification of the analyte.

Thus, in one preferred embodiment, the present invention provides a process of detecting the presence and determining the quantity of an analyte in a fluid suspected of containing the analyte, using a luciferase-mediated bioluminescence in a heterogeneous binding assay.

In a first such procedure, the fluid is contacted with a first binding partner specific for the analyte and immobilized onto an appropriate solid phase. The first binding partner specifically binds the analyte to form an analyte-first binding partner complex. This so-formed complex is contacted with a second binding partner having appropriate selectivity towards the analyte and to which is directly conjugated a luciferase enzyme to form a first binding partner-analyte-second binding partner complex. The excess luciferase-conjugated second binding partner is separated from the formed first binding partner-analyte-second binding partner complex, by standard, physical means e.g. by washing, and the formed complex is mixed with all components needed for the luminescent reaction including the photolabile trigger compound. Then the mixture is irradiated with a pulse of photonic energy, such pulse chosen with certain characteristics with regard to duration and wavelength, such that the said pulse detaches the trigger compound in active form from the caged compound, allowing the trigger compound to interact with the luciferase attached to analyte-binding partner complex and to cause luminescent emission from the complex. The photonic emission so produced creates a measurable bioluminescent photonic signal that can be detected and analyzed to determine analyte presence and concentration.

Another preferred embodiment of the invention utilizes an indirect luciferase-mediated bioluminescent assay to detect the presence of or to quantify an analyte in a fluid. This procedure comprises contacting the fluid with a first binding partner which is immobilized onto an appropriate solid phase, so that the first binding partner binds specifically to the analyte, to form an analyte-first binding partner complex. Then the said analyte-first binding partner complex is contacted with a second binding partner that specifically binds to the analyte to form a first binding partner-analyte-second binding partner complex. Excess second binding partner is separated from the first binding partner-analyte-second binding partner complex so formed, followed by addition to the said formed complex of a third binding partner to which is directly conjugated a luciferase enzyme and which binds specifically to the second binding partner to form a first binding partner-analyte-second binding partner-third binding partner complex. The excess third binding partner is separated from the so-formed complex, and the formed complex is mixed with the additional components needed for the luminescent reaction, including the photolabile trigger compound. Then the mixture is irradiated with a pulse of photonic energy, such pulse chosen with certain characteristics with regard to duration and wavelength, such that the trigger compound detaches in active form from the caged compound, allowing the trigger compound to interact with the luciferase attached to analyte-binding partner complex and to cause luminescent emission from the complex. The photonic emission so produced creates a measurable bioluminescent photonic signal that can be detected and analyzed to determine analyte presence and concentration.

In another preferred embodiment, the present invention provides for a process of detecting and quantifying the presence of an analyte in a fluid suspected of containing said analyte by carrying out a separation competition assay utilizing the luciferase bioluminescent reaction triggered by a photoreleased trigger compound released from a photolabile caged compound. According to such preferred embodiment, an analog of the target analyte is conjugated to a luciferase enzyme and competes with the analyte for binding to a specific binding partner which is immobilized to a solid phase in order to facilitate separation of bound and unbound fractions. The analyte analog competes with the analyte for binding to a limited amount of the specific binding partner to form a specific binding partner-analyte complex or specific binding partner-analyte analog complex. The bound and unbound fractions are separated and the luciferase-mediated bioluminescent reaction is triggered by the photorelease of a caged trigger compound either in the bound or unbound fractions and therefore the presence and concentration of the analyte is detected.

In yet another preferred embodiment, the process of the invention provides for a non-separation energy transfer assay utilizing the luciferase bioluminescent reaction triggered by a photoreleased trigger compound released from a photolabile caged compound. In such a procedure of detecting the presence of an analyte in a fluid, the analyte is bound to a first binding partner and a second binding partner to form a first binding partner-analyte-second binding partner complex. The first binding partner is conjugated to a luciferase enzyme and the second binding partner is conjugated to a fluorescent compound. The photorelease of a caged trigger compound triggers a bioluminescent-mediated light generation by the luciferase enzyme. The luminescent signal so generated results in the excitation of the fluorescent compound conjugated to the second binding partner and light emission. The luciferase and the fluorescent compound are chosen such that the bioluminescent emission from the luciferase reaction is capable of exciting the fluorescent tag conjugated to the second binding partner. Only the fluorescent tag in the vicinity of the luciferase reaction is excited, so that the assay can be carried out without a separation step.

An example of such a combination of bacterial luciferases occurs when conjugating Renilla reniformis luciferase to a first binding partner and conjugating Renilla green fluorescent protein to a second binding partner. The luminescent emission generated by the Renilla luciferase excites the green fluorescent protein of Renilla. The triggered luciferase emits oxyluciferin in the vicinity of the fluorescent protein only when they are combined as a complex. The fluorescent protein only becomes excited when it is attached to the complex. Free luciferase also becomes excited, but does not undergo fluorescent emission. A further example is provided by conjugation of bacterial luciferase to first binding partner and the conjugation of the yellow fluorescence protein to the second binding partner.

In another preferred embodiment, the process of the invention provides a method for detecting receptor occupancy in a receptor-ligand binding assay using the competitive or non-competitive format. In such an assay, a ligand to a cell receptor is conjugated to a luciferase and is mixed with its respective cell receptor with or without the presence of a competing ligand. The bound receptor-ligand complex is separated and the luciferase bioluminescence is triggered in the bound or the non-bound fraction by photorelease of the caged trigger compound so as to release the trigger compound in active form, and the bioluminescence is measured.

The selected luciferase and the caged trigger compound for use in the present invention need to be chosen on the basis of their mutual specificity to cause luminescent emission. For example, if the chosen luciferase is the firefly luciferase enzyme, the caged trigger compound is a caged ATP or a caged luciferin. Other examples of appropriate trigger compounds for use in preferred embodiments of the present invention include caged NAD and NADP with bacterial luciferases.

The caged trigger compound, such as caged ATP or caged luciferin, is unable to trigger the luminescent reaction of the firefly luciferase despite the presence of all components needed for triggering the luminescent emission. However, upon photonic pulse stimulation, of specific wavelength, the resulting, substantially instantaneous release of active ATP or luciferin is able to trigger the luminescent reaction. In such case, all of the luminescent photonic emission is derived from the trigger compound released from the added inactive photolabile caged trigger compound. This allows for the accurate monitoring of the amount of analyte present in the sample as inferred from the amount of the luciferase enzyme in the analyte-binding partner complex.

Caged trigger compounds useful in the present invention include those utilizing various caging groups. Thus, the trigger compound may be chemically bound through a photolabile chemical link or bond to a carrier compound, or may be physically entrapped within a photolabile carrier. A wide variety of such bonds are known in the art, for example those groups and bonds used as photolabile protective groups in the chemical synthesis of peptides.

The term “caged compounds” was coined for photolabile derivatives of natural substrates such as ATP—see Kaplan, J. H., Forbush, G. and Hoffman, J. F. “Biochemistry”, Volume 17, pages 1920-1935 (1978). The necessary properties of successful caged groups are discussed by Givens, Richard S., Weber, Jorg F. W., Jong, Andreas H. and Park Chan-Ho, “Methods in Enzymology”, Volume 291, pages 1-29 (1998). The disclosures of both of these references are incorporated herein in their entirety.

For ATP, examples of suitable precursor compounds for use in the present invention as “caged ATP compounds”, include those which utilize the o-nitrobenzyl group, such as 2-nitrobenzyl ATP and 2-nitrophenethyl-ATP. Other suitable photolabile groups for ATP and other nucleotide-like trigger compounds include desyl (diphenylethylketonyl) and p-hydroxyphenacyl. Use can also be made of the photolabile groups employed as photocleavable affinity tags to permit subsequent photorelease of an affinity label from a target by a molecule—see Olejnik, Jerzy; Krzymanska—Olejnik, Edyta; and Rothschild, Kenneth J., “Methods in Enzymology” Volume 291, pages 135-154 (1998), the disclosure of which is incorporated herein by reference.

Such precursor ATP with various caging groups is commercially available from Molecular Probes Inc. (Eugene, Oreg.) as a disodium salt (DMNPE-caged ATP and NPE-caged ATP, catalog numbers A-1049 and A-1048 respectively). Also, a caged D-luciferin is commercially available (Catalog number L-7085, Molecular Probes (Eugene, Oreg.). With regard to bacterial luciferases, Cohen discloses in U.S. Pat. No. 6,020,480 two different sets of caged NAD and also NADP which employ DMNPE and CNB as the photolabile groups, and which can be used herein.

The trigger compound can also be physically trapped within a particle that upon a photonic pulse releases the trigger compound substantially instantaneously. Instead of a photolabile chemical bond, one can use a photolabile physical association of the trigger compound with a carrier compound, for example, as with a photolabile liposomal encapsulation of the trigger compound in a photosensitive lipid membrane as taught by Morgan C G (Morgan et al, Photochem Photobiol, Vol. 62, 24-29,1995) incorporated herein by reference. Also, such a photolabile carrier can be a photolabile dendimer as disclosed in the U.S. Pat. No. 5,795,581 (Segalman) incorporated herein by reference. The photolabile groups utilized in these two carriers are different.

The photorelease of the trigger compound in active form from the carrier compound should preferably be substantially instantaneous. This requires the choice of a suitable photolabile linkage between the trigger compound and the carrier compound, which in turn depends upon the chemistry of the respective individual components, and the application of incident radiation of an appropriate frequency and also on energy level to cause the required release.

The invention is further described with reference to the accompanying illustrative but non-limiting experimental examples.

The method of the invention was demonstrated by carrying out luciferase chemiluminescent reaction experiments with one of the essential components of the reaction being initially caged. Upon delivering a UV light pulse of enough power, the caged compound is uncaged, causing an instantaneous release of an active compound to trigger the reaction. A typical luciferase chemiluminescent reaction needs the following essential components; luciferase enzyme, luciferin, magnesium and ATP in the presence of oxygen to generate light according to the following reaction:

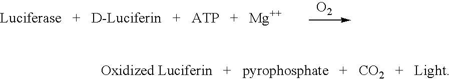

All components of the reaction are amenable to caging utilizing a photolabile bond which initially renders them inactive. The embodiment of the present invention of UV uncaging of an essential component to trigger a luciferase chemiluminescent reaction was demonstrated by carrying out the following examples.

### EXAMPLE 1

In this experiment, caged ATP was utilized to control the reaction. Functional ATP was delivered to the reaction from caged ATP upon exposure of the reaction components to a pulse of UV light. Reagents:

Lyophilized luciferase/lyophilized D-Luciferin were dissolved in Tricine buffer [50 mM N-tris(hydroxymethyl)methylglycine, adjusted with NaOH to pH 7.8, Lot 1418] both supplied by Kikkoman as assay kit, CheckLite HS Plus, Catalog # 60342).

5 mM Mg citrate in phosphate buffered saline (PBS, pH 7.4)

Caged ATP (p-(1-(4,5-dimethoxy-2-nitrophenyl)ether, disodium salt-caged adenosine-5-triphosphate) in methanol, 5 mg in 300 μL (Molecular probes, Catalog # A-1049).

In a total reaction volume of 20 μL, the following were mixed in a suitable reaction cell

10 μL of Luciferase/D-Luciferin solution mix,

5 μL of 5 mM Mg Citrate in PBS

5 μL of caged ATP solution.

The cell with the components was exposed to a pulse of UV (1 millisecond pulse by a Xenon Lamp, Rapp Optoelectronic). The power of the UV pulse was varied (40, 60, 80 mJ per pulse). The amount of light released from the reaction increased with the increase of the UV pulse power. In this reaction, ATP was the limiting factor and with increase of the UV pulse power, the amount of released ATP increased and therefore the amount of released light increased.

### EXAMPLE 2

In this experiment, caged D-Luciferin was utilized to control the reaction.

Functional D-Luciferin was delivered to the reaction from caged D-Luciferin upon exposure of the reaction components to a pulse of UV light with suitable power.

Reagents:

Firefly luciferase enzyme dissolved in Tricine buffer pH 7.8 (50 mM N-Tris (hydroxymethyl) methylglycine) adjusted with NaOH, supplied by Kikkoman Catalog LUC T

5 mM Mg citrate in PBS, pH 7.4-

1-(4,5-dimethoxy-2-nitrophenyl)ethyl ester—caged D-luciferin, 5 mg dissolved in 300 μL of dimethylsulfoxide DMSO, from Molecular Probes, Catalog # L-7085).

100 mM ATP solution, pH 7.5 (Amersham Pharmacia, Catalog No. 272056).

In a total reaction volume of 25 μL, the following components were added as solutions to a suitable cell:

10 μL of luciferase solution

5 μL of 5 mM Mg citrate in PBS

5 μL of 1 mM ATP solution

5 μL caged D-Luciferin solution.

The cell with the components was exposed to a pulse of UV (1 millisecond_pulse by a Xenon Lamp, Rapp Optoelectronic). The power of the UV pulse was varied (40, 60, 80 mJ per pulse). The amount of light released from the reaction increased with the increase of the UV pulse power. In this reaction, D-Luciferin was the limiting factor and with increase of the UV pulse power, the amount of released active D-Luciferin increased and therefore the amount of released light increased.

In addition, in both of the above experimental examples, multiple pulses of the same power were used (40 mJ per pulse). In this case, the first pulse resulted in the limited release of the caged components, which triggered the initiation of the luciferase chemiluminescent reaction and the emission of light. A second pulse resulted in a spike of the intensity of the steadily emitted light.

1. A process of inducing luminescent emission from a luciferase bioluminescent reaction mixture which comprises:
preparing a reaction mixture including components required for a specific luciferase bioluminescent reaction, said components including a luciferase, a luciferin chosen for specific interaction with said luciferase, and at least one cofactor therefor, one said component being initially included in the reaction mixture in an inactive, caged form;
subjecting the reaction mixture to photonic radiation of appropriately chosen characteristics to cause release of the one said component from its caged form, in active form thereby triggering the bioluminescent reaction;
and detecting the photonic emission so caused.

2. The process of claim 1 wherein the reaction component initially in caged form is a cofactor.

3. The process of claim 1 wherein the reaction component initially in caged form is the luciferin.

4. The process of any preceding claim wherein the luciferase is firefly luciferase, bacterial luciferase or fungal luciferase.

5. The process of any preceding claim wherein the cofactor is selected from adenosine triphosphate (ATP), nicotinamide-adenine dinucleotide phosphate (NADP), nicotinamide-adenine dinucleotide (NAD) and flavin mononucleotide (FMN).

6. The process of claim 4 wherein the luciferase is firefly luciferase and the luciferin is D-luciferin.

7. The process of claim 6 wherein the caged compound is caged ATP.

8. The process of claim 6 wherein the caged compound is caged D-luciferin.

9. The process of any preceding claim wherein the reaction mixture is utilized to detect the presence of an analyte in a biological fluid by binding reactions.

10. The process of claim 9 wherein the reaction mixture detecting the presence of an analyte in a biological fluid further includes a first binding partner capable of specifically binding the analyte and immobilized on a solid phase, and a second binding partner having appropriate selectivity towards the analyte and to which the luciferase is conjugated, so as to utililize the bioluminescence reaction initiated by release of the caged compound in active form to assay the analyte.

11. The process of any of claims 1-8 wherein the reaction mixture further includes an analyte under analysis therein, a first binding partner capable of specifically binding the analyte and immobilized on a solid phase, thereby forming an analyte-first binding partner complex, a second binding partner that specifically binds to the analyte to form a first binding partner-analyte-second binding partner complex, and a third binding partner to which the luciferase is conjugated and which binds specifically to the second binding partner to form a first biding partner-analyte-second binding partner-third binding partner-luciferase complex, so as to utilize the bioluminescence reaction initiated by release of the caged compound in active form to assay the analyte.

12. A process of inducing luminescent emission from a luciferase bioluminescent reaction, which comprises subjecting a luciferase/luciferase emitter, in the presence of inactive, caged, appropriately chosen trigger compound, to photonic radiation adapted to release the trigger compound in active form, and causing substantially instantaneous reaction of the active trigger compound so released with the luciferase-luciferase emitter so as to induce photonic emission therefrom.

13. A composition of matter including a luciferase/luciferase emitter, and a trigger compound which is a photolabile, caged cofactor adapted to release the cofactor in active form upon appropriate photonic energy input thereto, and being chosen to interact with the luciferase/luciferase emitter combination to initiate bioluminescence when the cofactor is photonically released in its active form.

14. A process according to claim 1 of detecting the presence of an analyte in a fluid suspected of containing said analyte, as applied to a heterogeneous binding assay, which comprises:
contacting the fluid with a first binding partner specific for the analyte, said first binding partner being immobilized onto an appropriate solid phase, and said first bind partner specifically binding the analyte to form an analyte-first binding partner complex;
contacting the so formed complex with a second binding partner to form a first binding partner-analyte-second binding partner complex, said second binding partner having appropriate selectivity towards the analyte, and said second binding partner being directly conjugated to a luciferase enzyme;
separating the excess luciferase-conjugated second binding partner from the so formed complex;
mixing the formed complex with all remaining components needed for the luminescent reaction including the photolabile trigger compound;
irradiating the mixture with a pulse of photonic energy, such pulse chosen with certain characteristics with regard to duration and wavelength, such that the said pulse detaches the trigger compound in active form from the caged compound;
allowing the trigger compound to interact with the luciferase attached to the analyte-binding partner complex and to cause luminescent emission from the complex, the emission so produced creating a measurable bioluminescent signal; and
detecting the emitted photons so produced.

15. A process according to claim 1 of detecting the presence of an analyte in a fluid suspected of containing said analyte by carrying out an indirect luciferase-mediated bioluminescent assay, which comprises:
contacting the fluid with a first binding partner immobilized onto an appropriate solid phase, said first binding partner binding specifically to the analyte to form an analyte-first binding partner complex;
contacting the said analyte-first binding partner complex with a second binding partner, said second binding partner specifically binding to the analyte to form a first binding partner-analyte-second binding partner complex;
separating excess second binding partner from the first binding partner-analyte-second binding partner complex so formed; adding to the so formed complex a third binding partner which is directly conjugated to a luciferase enzyme and which binds specifically to the second binding partner to form a first binding partner-analyte-second binding partner-third binding partner complex;
separating the excess third binding partner from the so-formed complex;
mixing the so-formed complex with all the remaining components needed for the bioluminescent reaction including the photolabile trigger compound;
irradiating the mixture with a pulse of photonic energy, said pulse being chosen with certain characteristics with regard to duration and wavelength such that the said pulse detaches the trigger compound in active form from the caged compound;
allowing the trigger compound to interact with the luciferase attached to the analyte-binding partner complex and causing luminescent emission from the complex, the emission so produced creating a measurable bioluminescent photonic signal; and
detecting and analyzing the emitted photons.

16. A process according to claim 1 of detecting the presence of an analyte in a fluid suspected of containing said analyte by carrying out a separation competition assay utilizing the luciferase bioluminescence reaction triggered by a photoreleased trigger compound released from a photolabile caged compound, which comprises:
conjugating an analog of the target analyte to a luciferase enzyme and contracting the conjugate with a specific binding partner immobilized onto a solid phase, so that the analyte analog competes with the analyte for binding to a limited amount of the specific binding partner to form a specific binding partner-analyte complex or specific binding partner-analyte analog complex;
separating the bound and unbound fractions utilizing the solid phase;
adding the remaining components needed for the luciferase-mediated bioluminescent reaction including a photolabile caged trigger compound and the triggering reaction by the photorelease of the caged trigger compound either in the bound or unbound fractions so as to detect the presence and concentration of the analyte.

17. A process according to claim 1 of detecting the presence of an analyte in a fluid suspected of containing said analyte by carrying out a non-separation energy transfer assay utilizing the luciferin-luciferase bioluminescence reaction triggered by a photoreleased trigger compound released from a photolabile caged compound, which comprises:
binding the analyte to a first binding partner and a second binding partner to form a first binding partner-analyte-second binding partner complex, the first binding partner being conjugated to a luciferase enzyme and the second binding partner being conjugated to a fluorescent compound;
photoreleasing a caged trigger compound to trigger a bioluminescence-mediated light generation of the luciferase, so as to cause excitation of the fluorescent compound conjugated to the second binding partner and consequent light emission; and
detecting said light emission.

18. A process according to claim 1 of detecting receptor occupancy in a receptor-ligand binding assay using the competitive or non-competitive format, which comprises;
conjugating to a luciferase a ligand to a cell receptor;
said ligand being mixed with its respective cell receptor with or without the presence of a competitor ligand;
separating the bound receptor-ligand complex,
adding luciferase-reacting reagents including the caged trigger compound to the receptor-ligand complex;
initiating the luciferase bioluminescence in the bound or the non-bound fraction by photorelease of the caged trigger compound so as to release the trigger compound in active form; and measuring the bioluminescence photons emitted.
measuring the bioluminescence photons emitted.
